(19) United States
(12) Patent Application Publication
(10) Pub. No.: US 2018/0271572 A1
(43) Pub. Date: Sep. 27, 2018

(54) MINIATURE OR MICRO-SCALE CONFORMABLE CHAIN MAIL DEVICE FOR ORTHOPAEDIC FIXATION, STABILIZATION, AND REPAIR

(71) Applicant: SUNNYBROOK RESEARCH INSTITUTE, TORONTO (CA)

(72) Inventors: Cari Marisa Whyne, TORONTO (CA); Jacob Zachary Fishman, NORTH YORK (CA)

(21) Appl. No.: 15/936,799

(22) Filed: Mar. 27, 2018

Related U.S. Application Data
(60) Provisional application No. 62/477,130, filed on Mar. 27, 2017.

(51) Int. Cl. A61B 17/80; A61F 2/28

(52) U.S. Cl. CPC A61B 17/8085; A61F 2002/3006; A61F 2/2875

(57) ABSTRACT

The present application provides miniature and micro-scale conformable chain mail devices for skeletal fixation, stabilization, and repair, and methods of manufacture and use thereof. The structural devices comprise a conformable sheet of interconnecting polygonal links that form a chain mail mesh having a first and a second outer surface, wherein: the interconnecting links comprise planar surfaces that combine to form the first and second outer surfaces, respectively, of the conformable sheet. Also provide are methods of using the structural device for stabilization of bone tissue, for fixation of bone tissue, as a bone graft patch or as a thin bone tissue replacement.

### FIELD

The present application pertains to the field of miniature and micro-scale devices and materials. More particularly, the present application relates to miniature and micro-scale devices and materials useful for bone fixation, stabilization and repair, and methods of manufacture and uses thereof.

### BACKGROUND

Implants for use in stabilizing adjacent bony structures facilitate bone healing by maintaining the adjacent bony structures in a predetermined spatial relationship, while new bone is formed connecting the fragments. Current bone fixation techniques rely predominantly on metal plates and screws to create immobilization in order to enable bone healing. Plates and screws can be a nidus for infection, requiring subsequent hardware removal, and result in issues with temperature sensitivity and palpability. In addition, the complex bone geometry often requires contouring of such implants to achieve adequate fixation. A high degree of conformability is required in order to mimic the natural curvature of complex bone structures as found in the craniomaxillofacial skeleton (CMFS). Difficulties also arise in achieving adequate screw purchase in the very thin bones of the CMFS.

Successful reconstructive procedures to heal CMFS, and other skeletal injuries, relies on accurate reduction and internal stabilization of bone fragments with complex morphologies in 3D space. A good fixation technique for the CMFS would therefore be a biocompatible, bioresorbable, low profile system that bonds to the surface of bone, remains flexible enough to allow for semi-stabilized accurate reduction of bone fragments in 3D space at multiple sites, and can then be fixed in place to stabilize bone fragments.

There remains a need, therefore, for a bone fixation, implantable device or material that addresses the drawbacks associated with current devices, such as plates and screws.

The above information is provided for the purpose of making known information believed by the applicant to be of possible relevance to the present invention. No admission is necessarily intended, nor should be construed, that any of the preceding information constitutes prior art against the present invention.

### SUMMARY

An object of the present application is to provide miniature and micro-scale conformable chain mail devices for skeletal fixation, stabilization, and repair, and methods of manufacture and use thereof. In accordance with an aspect of the present application, there is provided a structural device comprising a conformable sheet of interconnected non-planar polygonal links that form a chain mail mesh having a first upper and a second lower outer surface, and having a sheet thickness of 5 mm or less. Each interconnected non-planar polygonal link comprises planar surfaces that combine to form the first and second outer surfaces of the mesh which when said chain mail mesh is placed on a flat surface said first upper and second lower outer mesh surfaces are completely planar, respectively, of the conformable sheet. The device is suitable: for stabilization of bone tissue; for fixation of bone tissue; as a bone graft patch; as a thin bone tissue replacement; or any combination thereof. Optionally, the interconnected links are manufactured from biocompatible or bioresorbable plastic, metal or ceramic.

In certain embodiments, the interconnecting links of the chain mail mesh comprise at least two lower horizontal connector members (or beams), which define one of said planar surfaces, attached to at least two upper horizontal connector members (or beams), which define the other of said planar surfaces, at vertical corner posts to form a continuous link having a central opening. The connector beams can each have a thickness in the range of from about 0.2 mm to about 3 mm, or from about 0.2 mm to about 0.8 mm.

The interconnecting links can each be, for example, square shaped, rectangular, triangular, pentagonal, hexagonal or pyramidal. In some examples, the chain mail mesh comprises a combination of interconnecting links of two or more different shapes.

In accordance with one embodiment, the conformable sheet is adapted so that the device can be attached to a material, such as bone, using adhesive, or one or more screws, or a combination of one or more screws and adhesive.

The chain mail mesh may have a thickness of from about 0.4 to about 5 mm, from about 2 mm or less, or from about 0.6 mm to about 2.0 mm.

In some embodiments, the structural device, or the chain mail mesh sheet of the structural device, is manufactured using additive manufacturing (or a 3D printing method).

In some embodiments, the chain mail mesh of the structural device comprises an outer, interconnected border of polygonal end cap links surrounding a perimeter of the mesh, said polygonal end cap links being narrower than interior links surrounded by said end caps.

In some embodiments, the device is embedded as a structural reinforcing element within a polymer composite.

In some embodiments, the device additionally comprises an adhesive or other means for fastening the device to another material (e.g., bone)

In some embodiments, the device additionally comprises an elastic polymer phase within or between the interconnecting links.

In accordance another aspect of the present application, there is provided an implant comprising a polymer composite and a structural reinforcing element, wherein the structural reinforcing element is a device as described herein.

In accordance another aspect of the present application, there is provided a method of bone stabilization comprising: attaching a structural device as described herein to at least two adjacent bony structures, such as, for example, bony structures in a craniomaxillofacial skeleton. In some embodiments, the method additionally comprises conforming the device to an anatomical shape, by taking advantage of the conformability of the chain mail mesh. Depending on the material used to manufacture the device, it may be bioresorbed following bone growth between the stabilized adjacent bony structures.

In some embodiments of this method, the structural device is used for: conformable reinforcement to improve strength in cranioplasty implants with patient-specific contouring; or for sealing and/or patching bone graft packing (for example, by attaching the device to edges of the at least two adjacent bony structures and positioned over a grafting area).

### BRIEF DESCRIPTION OF THE DRAWINGS

For a better understanding of the application as described herein, as well as other aspects and further features thereof, reference is made to the following description which is to be used in conjunction with the accompanying drawings, where:

FIGS. 1A, 1B and 1C depict an embodiment of a single chain link with an overall square profile and having two top connector beams (12), the two bottom connector beams (14), and four corner posts (16) attaching the connector beams in flat-bottom U-shapes or inverted U-shapes to form the complete linkage. The completely flat or planar upper outer surface of the top connector beam is also depicted (12A) and the completely flat lower outer surface of the bottom connector beams are underneath. FIG. 1A is a front perspective view of the single chain link, FIG. 1B is a side perspective view of the single chain link, and FIG. 1C is an isometric view of the single chain link.

FIGS. 2A and 2B depicts two interconnect links, each link having an overall square profile as shown in the embodiment of FIG. 1, where FIG. 2A is a top view and FIG. 2B is an isometric view of the interconnected links. The upper and lower connecting beams of one link (18) are free to move around the lower and upper connecting beams of its adjacent link (20), respectively. The chain mail mesh is interconnected in a 4-on-1 arrangement. FIG. 2A illustrates the horizontal spacing (22) between the connection beams of two adjacent links.

FIG. 3A depicts additional interconnected links, each link (24) having an overall square profile as shown in the embodiment of FIGS. 1A to 1C, forming a chain mail mesh sheet. The chain mail sheet illustrates the overall completely flat top surface (26) and bottom surface generated by the completely flat connector beams of the link design.

FIG. 3B illustrates the narrower end cap link component (28) added along the perimeter of the interconnected links.

FIG. 4A depicts isometric views comparing a square profile link, as embodied in FIG. 1.

FIG. 4B depicts an end-cap link design for these square links. The end-cap link has a narrower width (48) than the regular repeating square link, and is located at the perimeter edges of the mesh sheet to prevent tangling.

FIG. 5 depicts three examples of the chain mail design of a U-shaped link connector segment with internal angled overhangs (30) at different angles and the effect on overall thickness. The internal overhang forms a bridge element (32) for the top connector beams that can be additive manufactured without the use of an internal support structure.

FIG. 6 depicts an embodiment of the chain mail design with an overall triangular profile. In this embodiment, there are three top (34) and three bottom connecting beams (36), connecting beams, each connected to each other via U-shaped link sections. There are three corner posts (38) and three mid-link (40) posts to attach them to complete the linkage.

FIG. 7 depicts interconnected links, each having an overall triangular shape (42) as depicted in the embodiment of FIG. 5, forming a chain mail mesh sheet. The chain mail sheet illustrates the overall completely flat top surface (44) and bottom surface generated by the flat connector beams of the link design. The triangle profile possesses larger openings between links (46) and different conformability than the square profile embodiment.

### DETAILED DESCRIPTION

### Definitions

Unless defined otherwise, all technical and scientific terms used herein have the same meaning as commonly understood by one of ordinary skill in the art to which this invention belongs.

As used in the specification and claims, the singular forms “a”, “an” and “the” include plural references unless the context clearly dictates otherwise.

The term “comprising” as used herein will be understood to mean that the list following is non-exhaustive and may or may not include any other additional suitable items, for example one or more further feature(s), component(s) and/or ingredient(s) as appropriate.

As used herein, the term “exemplary” means “serving as an example, instance, or illustration,” and should not be construed as preferred or advantageous over other configurations disclosed herein.

As used herein, the terms “about” and “approximately,” when used in conjunction with ranges of dimensions of components, particles, compositions of mixtures, or other physical properties or characteristics, are meant to cover slight variations that may exist in the upper and lower limits of the ranges of dimensions so as to not exclude embodiments where on average most of the dimensions are satisfied but where statistically dimensions may exist outside this region. It is not the intention to exclude embodiments such as these from the present disclosure.

As used herein, the phrase “completely flat” means globally flat or planar when on a flat surface and each link upper and lower outer surfaces are completely flat to adhere to its local bonding sites.

As used herein, in reference to use of the present device for skeletal fixation, stabilization and/or repair, the term “flexible” means conformable to the contours of bone and allows for required motion to align multiple bone fragments associated with a given fracture site.

As used herein, the term “craniomaxillofacial skeleton,” or “CMFS,” is used to refer to the anatomical area of the jaw, facial bones, skull, as well as associated structures.

As used herein, the term “biocompatible” means the material will have no adverse effects on cells, tissue or function in vivo, or in other words, it is biologically and physiologically compatible.

As used herein, the term “bioresorbable” means once broken down, the material will be assimilated over time naturally within the in vivo physiologic environment.

The present application provides a device suitable for use as an implant for bone fixation, for example, in orthopaedic, plastic or oral surgeries. The device comprises a plurality of interconnecting links, which together form a strip or sheet of chain mail mesh. The device can be implanted in a subject (e.g., a human patient) and is conformable to complex contours, such as those of human skeletal structures.

The device provided herein takes advantage of properties associated with chainmail. As is well known, chain mail is made from a series of interlocking or interconnecting links. The most widely known use of chain mail was as armour, which was made from small metal rings linked together in a pattern to form a mesh-like material. The interlocking rings of the chain mail armour provided the wearer protection from weapons without severely limiting their movement, as occurred with plate armour. The chain mail structure in the present device differs from a standard, armour-type chain mail such that it is particularly suitable for use in applications that require fixation that allows for both strength and flexibility/conformability. The chain mail mesh described herein has been designed such that it is also suitable for manufacture using additive manufacturing (AM) techniques (also known as 3D printing techniques).

The presently provided device comprising the chain mail mesh strip or sheet can be used in orthopaedic, plastic and oral surgery for bone fixation, stabilization and repair. For implantation, the interlocking links are fabricated with additive manufacturing using biocompatible, or biodegradable/resorbable materials.

Chain Mail/Link Design

Generally, the strength and flexibility of chain mail is determined by three main factors: link type, link thickness, and inter-link spacing. The link material dictates the manufacturability associated with the link design's thickness and spacing. In the device of the present application, the chainmail has been designed taking into consideration each of these factors, as well the requirements of the application of the device, for example, as a bone stabilization device.

Thickness

To be suitable for implantation and bone stabilization, the interlocking links have a low to medium profile height (for example, <5 mm, <3 mm, <2 mm, about 0.4 mm to about 5.0 mm, or about 0.4 mm to about 2 mm) to minimize interference with surrounding tissue. In addition, each link is made comprising completely flathorizontal top and bottom surfaces, in order to maintain an overall smooth surface in the device sheet or strip comprised of many links. Each link is also made comprising completely flat, vertical side surfaces, rather than round or significantly curved surfaces. The link's flat side surfaces help to minimize potential tangling of adjacent links.

In order to increase the links per area, which increases the flexibility of the chainmail, the wall thickness of the links is minimized. In one embodiment, the link's wall thickness is from about 0.2 mm to about 1 mm, or from about 0.2 mm to about 0.8 mm. The actual wall thickness selected will depend on various factors, such as method of manufacture (see below), application, and the material used to construct the device.

Link Type

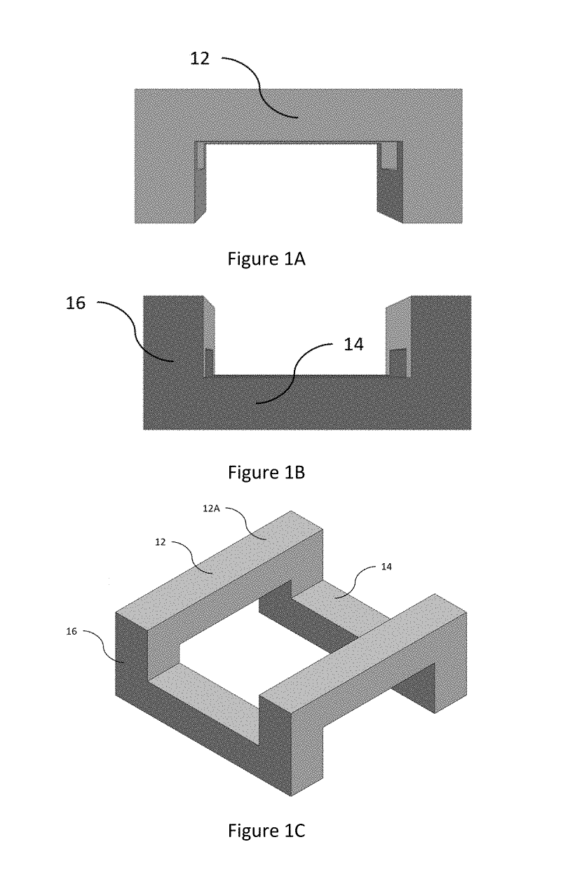

Each link in the present device comprises “upper” beam connections forming a bridge element and “lower” beam connections providing a support base. As would be well understood by a worker skilled in the art, the terms upper and lower are used to indicate the relative locations of the two beam connections and are not intended to refer to the absolute position of the connections as being above or below the other; the actual positioning of the connections will change as the device is moved. As illustrated by the embodiment shown in FIGS. 1A-C, each link comprises completely flat upper connecting beams (12) joined to lower connecting beams (14) at corner posts (16) to form a continuous polygonal shape having an interior opening defined between the upper and lower beams and connecting corner posts. The upper connecting beams are spaced apart from the lower connecting beams in parallel planes (non-coplanar).

As depicted in FIGS. 1A-1C, in one embodiment, each link is generally a square shape defined by cuboid connector beams. FIG. 1A is a front view of a square shaped link showing the outside face of a upper connecting beam (12) and two corner posts, which together form an upside down flat bottom U-shape. The completely flat or planar upper outer surface of the top connector beam is also depicted (12A) and the completely flat lower outer surface of the bottom connector beams are underneath. FIG. 1B is a side view of the square shaped link shown in FIG. 1A. As depicted in FIG. 1B, the lower connecting beam (14) together with the two corner posts (16) form a U-shape. FIG. 1C is isometric view of the square shaped link of FIGS. 1A and 1B. The upper (12) and lower (14) connecting beams shown in FIGS. 1A-1C are cuboids having equivalent depths, however, in some embodiments, the depth of the upper and lower connecting beams can differ.

In one non-limiting embodiment, to ensure a smooth surface in the chain mail mesh, the depth of the corner posts is equal to or greater than the sum of the depth of upper connecting beam(s) and the depth of the lower connecting beam(s). Use of the simple cuboid connecting beams improves AM of the chain mail mesh for small features sizes (<1 mm) which are at or near the printer's minimum wall thickness capability because a printing extrusion nozzle or curing/sintering laser movement, for example, can be performed using straight lines that are aligned to minimum step sizes of the printer's motors.

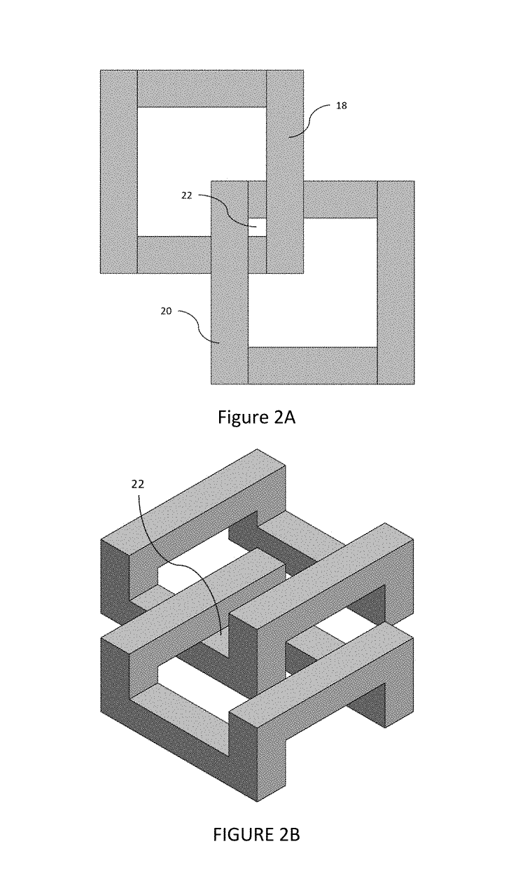

FIGS. 2A and 2B depict two interconnected links (18, 20), each having a structure as depicted in FIGS. 1A-1C. As shown in FIGS. 2A and 2B, the spacing between links (22), is the in-plane distance from a connecting beam of one link (18) to a connecting beam of another link (20). The spacing between adjacent links is selected such that two neighboring links are not printed too close together, and to avoid fusing links together, causing the chain mail mesh to become rigid. The link spacing must also not be too far apart, to avoid links that can flip over and tangle during use. In one embodiment, for AM with sterolithography (SLA) (detailed below), the spacing between links is between about 0.3 mm and about 0.5 mm. However, optimal spacing will scale proportionally with the wall thickness and will change depending on the manufacturing technique.

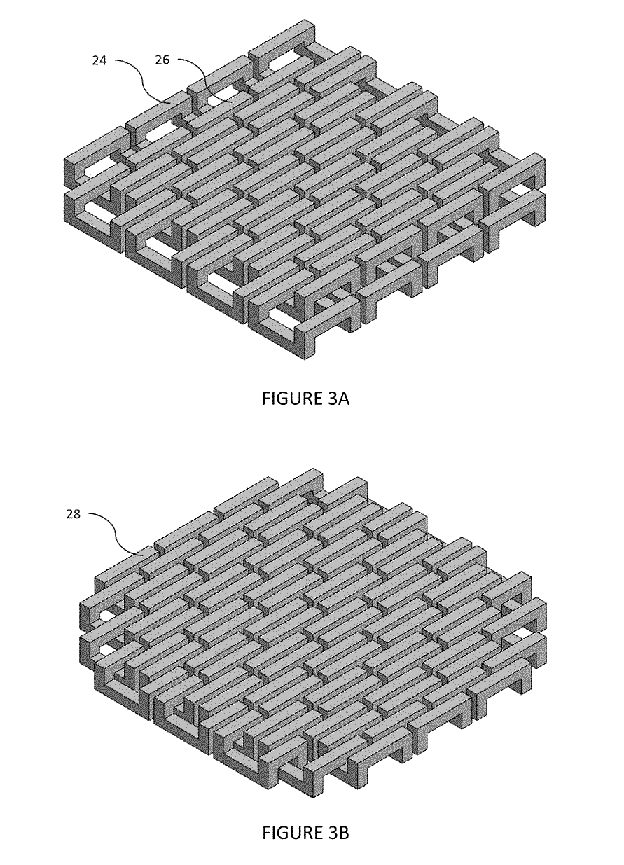

FIG. 3A depicts an example of a chain mail mesh sheet comprising links, according to the embodiment shown in FIGS. 1A-1C and 2A and 2B. The mesh is pliable, or conformable, because of the freedom of movement of each link (24) relative to its neighbouring interconnecting links. An overall completely flat upper and lower surface (26) for the mesh sheet is maintained as the mesh is conformed due to the many flat surfaces of each link's upper and lower connecting beams. The completely flat surfaces of a link's upper and lower connecting beams also provide more top and bottom contact surface area than a curved surface. This increased surface area from the upper or lower link and mesh surfaces is particularly useful in applications that require adhesion, for example, using glues or cements in application to bony tissue, but not limited to bony tissue (as further described in the Application of Conformable Device section below).

The range of movement for a link is determined, at least in part, by the amount of free space around the link (interior and exterior) to which it is connected. Accordingly, the link mobility can be reduced by decreasing this space. Link mobility, in turn, influences the mechanical compliance or pliability of the mesh. For instance, portions of the chain mail mesh can be made less compliant by decreasing an interior spacing distance of the link, thereby limiting the movement of the links within that portion of the mesh. That is, there is less open space in the interior of a link having a reduced inner and/or outer distance; thus, interconnected links that pass through this space are restricted in their movement. Alternatively, the thickness of each link can be increased, which also restricts the range of motion between interconnected links.

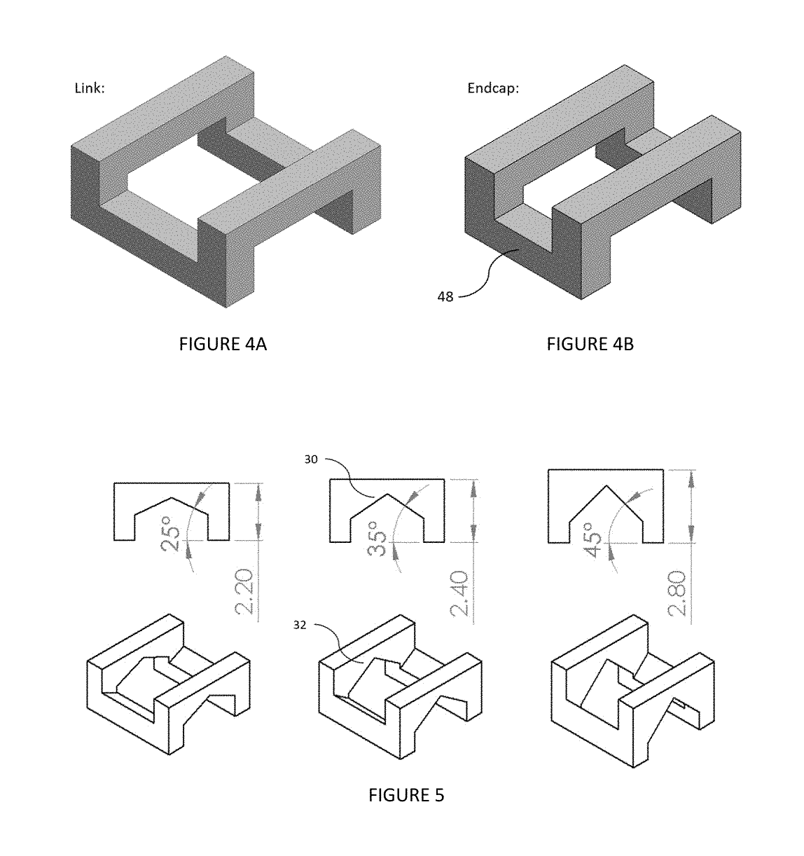

In one embodiment, the chain mail mesh incorporates narrower end cap link components (28) to form a border at the edge of the interconnected link mesh, as illustrated in FIG. 3B. These end cap links reduce the possibility that links will flip over at the edges due to excess spacing, which would interrupt the flatness of the mesh sheet. FIG. 4A provides an isometric view of a square repeating link (as also shown in FIG. 1C) in comparison to FIG. 4B, an isometric view of one example of an end cap link. In this example, the end cap is narrower (48) than the square repeating links and, consequently, limits the movement of adjacent, interconnected links so that they cannot flip over.

In one embodiment, the upper connecting beams of each link include internal sloped or arched faces to build the upside down or inverted U-shape connector bridge segment structure without additional support material underneath, as would be needed in some AM techniques (described below). For a chain mail mesh sheet with overall thickness <5.0 mm, internal support material would be a challenge to remove with additional machining operations. A secondary processing step, for example, with chemical etching, to remove internal support structure is also avoided with the internal angled bridge structure design. An example of this structure is shown in FIG. 5. As depicted in FIG. 5, each link is square-shaped, with an internal opening defined by two pairs of connecting beams joined at four corner posts, where each pair of connecting beams comprises two parallel beams. Similar to the embodiment shown in FIGS. 1A, 1B and 2A, 2B, the connecting beams provide parallel, spaced apart planar “upper” and “lower” surfaces. In this example, the underside of the connecting beams are sloped or arched so that it is self-supported in the material layer building process during manufacturing. As illustrated in FIG. 5, the overhang slope angle (30) impacts the overall thickness of the mesh, where decreasing the angle minimizes the overall thickness. In one embodiment, as shown in FIG. 5, the lower connecting beams comprise a sloped, upper face and the upper connecting beams comprise two lower faces that form an upside down “V” shape (32). The slope of each face of the upside down “V” is complementary to the slope the upper face of the lower connecting beam of the link with which it interacts. This configuration reduces the free space between adjacent links to reduce link rotation and tangling of the chain mail mesh.

In a particular embodiment of the device depicted in FIG. 3B, the mesh is for use in bone fixation, stabilization and/or repair. In this embodiment one of the upper or lower surfaces is for adhering to bone tissue, and the other of the upper or lower surfaces is the soft tissue facing side.

In alternative embodiments, each link is generally a triangular, rectangular, hexagonal or other polygonal shape. In each case, however, each link comprises at least one upper connecting beam and at least one lower connecting beam, with the total number of connecting beams being three or more, each connected at posts to form a polygonal shape. The use of different shaped links will produce chain mail mesh having different conformability characteristics. This allows the device to be tailored to particular uses, for example, uses that have certain anatomy-specified requirements.

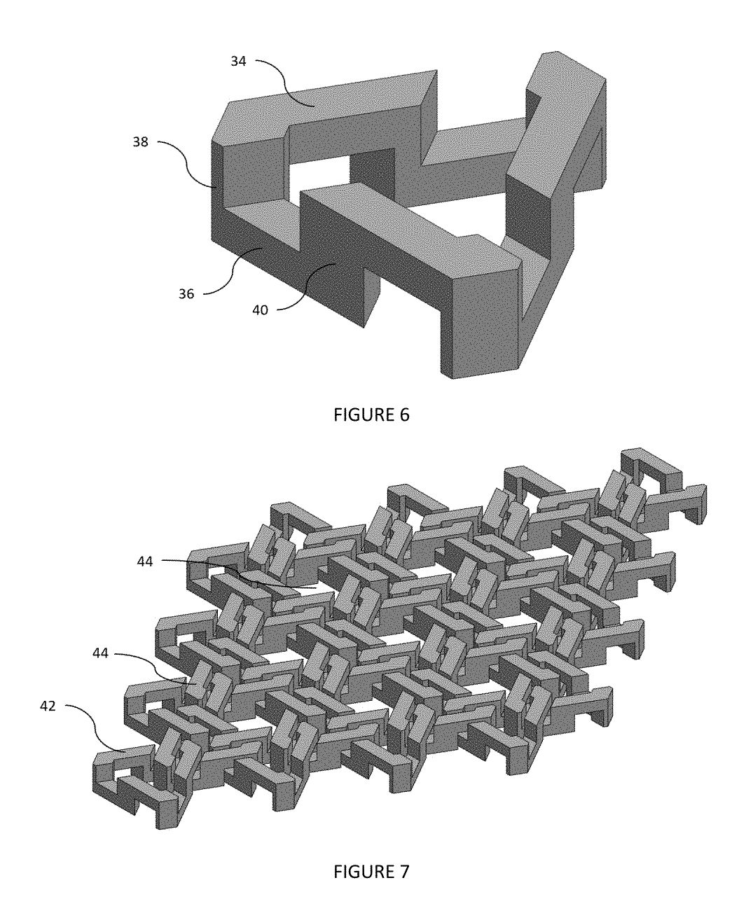

FIG. 6 depicts an example of a link structure suitable for use in the chain mail mesh of the present device. This link is triangular and comprises three upper connecting beams (34), three lower connecting beams (36) and six connecting posts (38, 40). Each side of the triangle comprising one upper connecting beam and one lower connecting beam so that an overall completely flat upper and lower surface is formed with interconnection of the links. As detailed above, the chain mail mesh shown in FIG. 6 is one in which each link has a low to medium profile height (for example, <5 mm, <3 mm, <2 mm, about 0.4 mm to about 5.0 mm, or about 0.4 mm to about 2 mm) and a connecting beam wall thickness from about 0.2 mm to about 3 mm, or from about 0.2 mm to about 0.8 mm. The chain mail links in this embodiment are also made from biocompatible or bioresorbable material.

FIG. 7 depicts another example of a chain mail mesh sheet comprising links (42) according to the embodiment shown in FIG. 6. The mesh is pliable, or conformable, because of the freedom of movement of each link relative to its neighbouring interconnecting links, however, a generally overall completely flat upper (44) and lower surface is maintained as the mesh is conformed due to the flat surfaces of the upper and lower connecting beams.

Alternative/Additional Components

The main component of the present conformable device is the chain mail mesh described above. However, it should be readily appreciated that the device can incorporate additional components. For example, the device can include one or more adhesives to facilitate attachment to bony structures during implantation. In an alternative embodiment, the chain mail mesh can have other parts or features, with non-link geometries, embedded. In one example, the chain mail mesh includes through-hole features distributed throughout, which provide a feature for screw fixation to thicker bone for attachment of the device during use. The chain mail mesh can also be embedded within an elastic polymer phase within or between the links to make a composite material for additional strength.

Material

The interconnecting links of the present device can be manufactured from a variety of materials, which can be selected based on the application of the device and the method of manufacture. In some embodiments, the device is manufactured using biocompatible or bioresorbable materials such as, but not limited to, plastics (e.g. Polylactic acid (PLA)), and metals (e.g., titanium). In other embodiments, the device is manufactured using bioresorbable materials, such as, but not limited to ceramics (e.g. calcium ortho-phosphates (e.g. tri-calcium phosphate, calcium poly-phosphate), calcium sulphates, and hydroxyapatite). These ceramic materials are commonly used in bone fillers and for grafting.

Manufacture

The device described herein can be manufactured using various techniques. In one embodiment, the device is manufactured using additive manufacturing (AM), colloquially known as 3D printing. AM processes enable fabrication of the chain mail mesh of the device in complete strips or sheets, without the need to interlock pieces one at a time. AM comprises a variety of technologies used to produce objects by building up sequential layers of material, based on a 3D computer aided design. The material types available for manufacturing the chain mail mesh are broadly in the categories: plastics, metals or ceramics. Each of these AM technologies are generally characterized by the process used for building up the layers, which is also associated with the material type used. For example, sequential layering with plastics is achieved through melting or curing; metals are layered via melting or sintering; and ceramics are glued and/or sintered.

In some embodiments, the device of the present application is fabricated using biocompatible or biodegradable/resorbable plastics (e.g., PLA, etc.) with AM processes, such as, but not limited to: fused deposition modeling (FDM); UV curing with stereolithography (SLA) or digital light processing (DLP); or selective laser sintering (SLS); or polyjet deposition techniques.

In alternative embodiments, the device of the present application is fabricated using biocompatible metals (e.g., titanium) with AM processes, such as, but not limited to: powder bed fusion, including direct metal laser sintering (DMLS) or selective metal melting (SLM) or electron beam melting (EBM); or metal binder jetting; or metal deposition including directed energy deposition (DED) or laser cladding; or metal infused composites.

In alternative embodiments, the device of the present application is fabricated using biodegradable/resorbable ceramics (e.g., calcium ortho-phosphates (e.g. tri-calcium phosphate, calcium poly-phosphate), calcium sulphates, and hydroxyapatite) with AM processes, such as, but not limited to: powder bed deposition including binder jetting or solid freeform fabrication (SFF); selective laser sintering (SLS); or lithography-based ceramic manufacturing (LCM), for the “green” part fabrication and the sintering post-process step.

In alternative embodiments, the device of the present application is fabricated using a negative mold which is produced via AM. A negative mold for ceramic casting may be comprised of a wax, plastic or paper and is ‘lost’ (via melt or burn off) prior to final sintering.

As should be readily appreciated, the wall thickness of each link's beams, the minimum spacing and the minimum overall thickness will vary depending on the method of manufacture and the material used. The overall thickness is a function of a link's wall thickness and spacing. The following table describes examples of the expected minimum wall and spacing thicknesses, depending on the printing technique and material used. Smaller thickness values are accessible, but push the limits of current technologies. Larger values represent currently readily achievable minimum wall thicknesses.

| | Min. Wall | Min. Spacing | Min. Overall |
|---|---|---|---|
| | Thickness (mm) | (mm) | Thickness (mm) |
| Plastic SLA/DLP | 0.6-0.8 | 0.3-0.4 | 1.5-2.0 |
| Metal DMLS | 0.3-0.4 | 0.3-0.4 | 0.9-1.2 |
| Ceramic LCM | 0.2-0.3 | 0.2-0.3 | 0.6-0.9 |

An exemplary device was printed in “Accura Clearvue” plastic with a SLA process on a 3D Systems Viper SI2. The exemplary chain mail link has a wall thickness of 0.7 mm and a spacing of 0.4 mm, which is near the minimum wall thickness using this printing technique. The overall thickness of the chain mail mesh sheet was 2×(vertical wall thickness)+vertical spacing=2×(0.7)+0.4=1.8 mm, composed of a lower beam connection, an upper beam connection, and the space between for clearance between the adjacent links. For this exemplary device, each link's interior distance was 2.80 mm, the outside distance was 4.40 mm and the center-to-center link spacing was 4.80 mm. Use of this system and material has been successfully employed to manufacture exemplary devices comprising the chain mail mesh as described herein, having square or triangular link shapes.

For this exemplary chain mail link to interconnect properly with its adjacent neighbour (FIG. 2A), the link outside distance length (L) is calculated a function of the inter-link horizontal spacing (Sₕ) and the link connecting beam horizontal wall thickness (Wₕ). This relationship can be expressed as L=4 Wₕ+3Sₕ, since a mesh must incorporate its own two (2) link beams, two (2) internal beams from its neighbours, the three (3) spaces between the four (4) beams. To determine the mesh patterning distance (d), the adjacent links are offset from the first link by one link's length and one additional spacing (d=L+Sₕ=4 Wₕ+4Sₕ). The link mesh pattern can be repeated in this way to increase or decrease the overall size of the mesh sheet, or to be increased or decreased uni-axially to generate a mesh strip.

To print the small features present in the chain mail mesh using additive manufacturing, it is beneficial to print the design without internal support structures that would hold up the link's upper connection to form the bridge. On the sub-millimeter scale, a post-processing mechanical clean-up step to remove the support structure between links is difficult or impossible. A secondary chemical etching step to remove internal support structure is possible to implement but is preferable to avoid.

Compared to other additive manufacturing techniques, SLA is able to print chain mail without internal supports between links because layers are added to the bottom of parts hanging upside-down. Therefore, the layers added to SLA printed plastic parts do not have to be designed to compensate for gravity. This is also applicable to parts printed using additive manufacturing in ceramic with LCM, however, incorporating angled overhangs can still improve the printing result.

Depending on the AM technique, the chain mail links are alternatively designed with internal angled or arched overhangs to avoid requiring a support structure, which would fuse links together. For printing with metal and ceramic without supports, the chain mail link's horizontal bridge (or upper connector beam) features can be replaced with an angled overhang. The overall thickness of the chainmail will then also depend on the minimum overhang achievable for a printing technique. This is depicted in FIG. 5, on a layer level and for varying angles on an individual link. The minimum overhang angle prescribed by the DMLS manufacturer design guidelines is 25°. However, the angle can be pushed down to as small as approximately 15° in order to minimize impact on thickness.

Application of Conformable Structural Device

The present application further provides methods of using the herein described conformable structural device for bone fixation, for example, as an implant in orthopaedic, plastic or oral surgical treatments. In one example, the conformable device is used in reconstructive surgery of the CMFS.

In some embodiments, the conformable device is used in combination with a biocompatible/bioresorbable adhesive for adhering the device to adjacent bony structures. In this embodiment, the conformable device is implanted over a portion of each of the bony structures with adhesive between the device and the bony structures so that the device is adhered in place. The device can be readily conformed to the required anatomical shape due to the pliability of the chain mail mesh. The completely flat upper and lower link surfaces very advantageously provide a large surface area for each link's adhesive contact with bony tissue. The conformable mesh comprised of the links therefore has a beneficial large surface area for bony tissue adhesion. The device can remain implanted or it can be bioresorbed over time.

In one embodiment, the conformable device is manufactured from a ceramic material. In one example of this embodiment, the ceramic, conformable device invention is used as the ceramic component in “Bone Tape” strips, sheets or other mesh configurations, to be embedded in a biodegradable flexible polymer and then adhered to stabilize fractures (see, International PCT Application PCT/CA2013/050570, which is incorporated herein by reference in its entirety).

In one example of this embodiment, there is provided an implantable device that comprises a composite flexible construct that includes a chain mail mesh sheet as described herein and a biocompatible cement on one surface of the chain mail mesh sheet. This composite flexible construct can be applied to bone such that the cement directly contacts the bone and can be cured to form a permanent bond between the bone and the construct. Optionally, the chain mail mesh sheet in the flexible construct is partially embedded in a polymer, such as a biodegradable and/or bioresorbable polymer.

In another embodiment, the conformable device is used for sealing and patching bone graft packing in place. In this embodiment, the chain mail mesh sheet of the device is adhered to the edges of bone, over the grafting area.

In another embodiment, the conformable device is used for conformable reinforcement to improve strength in cranioplasty implants with patient-specific contouring.

The characteristics of the conformable device that make it particularly suitable for the above orthopaedic, plastic and oral surgeries, also make the device useful in other applications. As such, the use of the conformable device is not restricted to orthopaedic, plastic and oral surgery applications. Non-limiting examples of alternative applications of the present conformable device are: reinforcing structural elements, such as in aerospace applications; body armour, such as in defence applications; sports padding (e.g., knee or shoulder pads); abrasion resistant layers in fabrics (e.g., motorcycle jackets); conformable layers in fabrics and textiles; and jewelry.

All publications, patents and patent applications mentioned in this Specification are indicative of the level of skill of those skilled in the art to which this invention pertains and are herein incorporated by reference to the same extent as if each individual publication, patent, or patent applications was specifically and individually indicated to be incorporated by reference.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

We claim:

1. A structural device, comprising:
a conformable sheet of interconnected non-planar polygonal links with each polygonal link able to move with respect to its neighboring links that form a chain mail mesh having a first upper and a second lower outer mesh surface, and having a sheet thickness of 5 mm or less;
each interconnected non-planar polygonal link comprises planar surfaces that combine to form the first and second outer surfaces of the mesh which when said chain mail mesh is placed on a flat surface said first upper and second lower outer mesh surfaces are completely planar, respectively, of the conformable sheet, and the device is suitable: for adhesion to bone tissue for stabilization of bone tissue and for fixation of bone tissue;
as a bone graft; and
as a bone tissue replacement, or any combination thereof.

2. The device of claim 1, wherein the chain mail mesh comprises an interconnected border of polygonal end cap links surrounding a perimeter of the mesh, said polygonal end cap links being narrower than interior links surrounded by said end caps.

3. The device of claim 2 wherein said end cap links and said interior links are composed of connector beam segments where all segments contain a flat-bottom U-shape or inverted U-shape.

4. The device of claim 1, wherein the interconnected links comprise at least two lower horizontal connector members, which define one of said planar surfaces, attached to at least two upper horizontal connector members, which define the other of said planar surfaces, at vertical corner posts to form a continuous link having a central opening.

5. The device of claim 4, wherein the thickness of each connector member is in the range of from about 0.2 mm to about 3 mm, or from about 0.2 mm to about 0.8 mm.

6. The device of claim 1, wherein the interconnecting links are square shaped, rectangular, triangular, pentagonal, hexagonal, pyramidal or any combination thereof.

7. The device of claim 1, wherein the sheet is for attachment to bone via an adhesive or screws.

8. The device of claim 1, wherein the mesh thickness is from about 0.4 to about 5 mm.

9. The device of claim 1, wherein the mesh thickness is about 2 mm or less, or from about 0.6 mm to about 2.0 mm.

10. The device of claim 1, wherein the interconnecting links are manufactured from biocompatible or bioresorbable plastic, metal or ceramic.

11. The device of claim 1, wherein the device is manufactured using additive manufacturing.

12. The device of claim 1, wherein the chain mail mesh comprises an outer, interconnected border of end cap links.

13. The device of claim 1, wherein the device is embedded as a structural reinforcing element within a polymer composite.

14. The device of claim 1, wherein the device additionally comprises an adhesive.

15. The device of claim 1, wherein the device additionally comprises an elastic polymer phase within or between the interconnecting links.

16. An implant comprising a polymer composite and a structural reinforcing element, wherein the structural reinforcing element is a device according to claim 1.

17. A method of bone stabilization comprising:
attaching a structural device of claim 1 to at least two adjacent bony structures to stabilize the adjacent bony structures.

18. The method of claim 17, additionally comprising:
conforming the device to an anatomical shape.

19. The method of claim 17, wherein the device is bioresorbed following bone growth between the stabilized adjacent bony structures.

20. The method of claim 17, wherein the bony structures are in a craniomaxillofacial skeleton.

21. The method of claim 20, wherein the structural device is used for conformable reinforcement to improve strength in cranioplasty implants with patient-specific contouring.

22. The method of claim 20, wherein the structural device is for sealing and/or patching bone graft packing.

23. The method of claim 22, wherein the device is attached to edges of the at least two adjacent bony structures and positioned over a grafting area.
